(19) United States
(12) Patent Application Publication
(10) Pub. No.: US 2009/0257983 A1
(43) Pub. Date: Oct. 15, 2009

(54) MEDICAL TREATMENT DEVICE FOR TREATING AIDS BY UTILIZING MODIFIED HUMAN IMMUNODEFICIENCY VIRUS VIRIONS TO INSERT ANTI-VIRAL MEDICATIONS INTO T-HELPER CELLS

(76) Inventors: Lane Bernard Scheiber, Annandale, VA (US); Lane Bernard Scheiber, II, Grosse Ile, MI (US)

Correspondence Address: LANE B. SCHEIBER, 7864 OAK RIVER DRIVE, GROSS ILE, MI, 48138, US

(21) Appl. No.: 12/101,958

(22) Filed: Apr. 11, 2008

(51) Int. Cl. A61K 31/7088; A61P 31/12; A61K 48/00

(52) U.S. Cl. 424/93.2; 514/44 R

(57) ABSTRACT

The medical device by which a modified Human Immunodeficiency Virus or virus-like structure is used as a transport medium to carry a payload of a quantity of anti-viral drug molecules to T-Helper cells in the body. The modified Human Immunodeficiency Virus or virus-like structure makes contact with a T-Helper cell by means of the modified virus's exterior probes or virus-like structure's exterior probes. Once the exterior probes engage the T-Helper cell's receptors, the modified virus or virus-like structure inserts into the T-Helper cell the quantity of medically therapeutic anti-viral drug molecules it is carrying. The anti-viral drug molecules exhibit an anti-viral effect when present inside the T-Helper cells thus assisting in repelling an infection by the Human Immunodeficiency Virus and the use of such a device significantly lowers the occurrence of unwanted deleterious side effects.

### CROSS-REFERENCE TO RELATED APPLICATIONS

None.

### STATEMENT REGARDING SPONSORED RESEARCH OR DEVELOPMENT

None.

### REFERENCE TO SEQUENCE LISTING, A TABLE, OR COMPUTER LISTING COMPACT DISC APPENDIX

Not applicable.

©2008 Lane B. Scheiber and Lane B. Scheiber II. A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

This invention relates to any medical treatment device intended to treat a medical condition in the body by utilizing a modified virus to insert a drug into cells of the body.

2. Description of Background Art

The approach to treating the AIDS epidemic has been to administer to patients anti-viral drugs to combat infection of T-Helper cells by the Human Immunodeficiency virus. This approach has produced limited success. Side effects of these anti-viral drugs has in some cases posed a deterrent to the success of such therapy.

Many drugs used to treat medical diseases have limited success and incomplete compliance by patients due to the fact that drugs often cause unwanted side effects when healthy cells suffer delirious effects of the drug. A drug introduced into the body by means of an oral route, inhaled route, rectal suppository or an injectable manner may affect every cell it comes in contact with rather than limiting its effects to the specific tissues or specific cells that the drug is intended to exert an effect on to generate a medically therapeutic outcome. Adverse side effects generated by systemic effects of drugs may be minimized by limiting the delivery of a drug to specific target cells or specific tissues in the body.

A eukaryote refers to a nucleated cell. Eukaryotes comprise nearly all animal and plant cells. A human eukaryote or nucleated cell is comprised of an exterior lipid bilayer plasma membrane, cytoplasm, a nucleus, and organelles. The exterior plasma membrane defines the perimeter of the cell, regulates the flow of nutrients, water and regulating molecules in and out of the cell, and has embedded into its structure cell-surface receptors that the cell uses to detect properties of the environment surrounding the cell membrane. Cytoplasm refers to the entire contents inside the cell except for the nucleus and acts as a filling medium inside the boundaries of the plasma cell membrane. Cytosol refers to the semifluid portion of the cytoplasm minus the mitochondria and the endoplasmic reticulum. The nucleus, organelles, and ribosomes are suspended in the cytosol. Nutrients such as amino acids, oxygen and glucose are present in the cytosol. The nucleus contains the majority of the cell's genetic information in the form of double stranded deoxyribonucleic acid (DNA). Organelles generally carry out specialized functions for the cell and include such structures as the mitochondria, the endoplasmic reticulum, storage vacuoles, lysosomes and Golgi complex. Floating in the cytoplasm, but also located in the endoplasmic reticulum and mitochondria are ribosomes. Ribosomes are protein structures comprised of several strands of proteins that combine and couple to a messenger ribonucleic acid (mRNA) molecule. More than one ribosome may be attached to a single mRNA at a time. Ribosomes decode genetic information coded in a mRNA molecule and manufacture proteins to the specifications of the instruction code physically present in the mRNA molecule.

The majority of the deoxyribonucleic acid (DNA) in a cell is present in the form of chromosomes, the double stranded helical structures located in the nucleus of the cell. DNA in a circular form, can also be found in the mitochondria, the powerhouse of the cell, an organelle that assists in converting glucose into usable energy molecules. DNA represents the genetic information a cell needs to manufacture the materials it requires to develop to its mature form, sustain life and to replicate. Genetic information is stored in the DNA by arrangements of four nucleotides referred to as: adenine, thymine, guanine and cytosine. DNA represents instruction coding, that in the process known as transcription, the DNA's genetic information is decoded by transcription protein complexes referred to as polymerases, to produce ribonucleic acid (RNA). RNA is a single strand of genetic information comprised of coded arrangements of four nucleotides: adenine, uracil, guanine and cytosine. The physical difference in the construction of a DNA molecule versus a RNA molecule is that DNA utilizes the nucleotide ‘thymine’, while RNA molecules utilize the nucleotide ‘uracil’. RNAs are generally classified as messenger RNAs (mRNA), transport RNAs (tRNA) and ribosomal RNAs (rRNA).

Proteins are comprised of a series of amino acids bonded together in a linear strand, sometimes referred to as a chain; a protein may be further modified to be a structure comprised of one or more similar or differing strands of amino acids bonded together. A protein comprised of one or more strands of amino acids (referred to as subunits) may be referred to as a protein complex. Insulin is a protein structure comprised of two strands of amino acids, one strand comprised of 21 amino acids long and the second strand comprised of 30 amino acids; the two strands attached by two disulfide bridges. There are an estimated 30,000 different proteins the cells of the human body may manufacture. The human body is comprised of a wide variety of cells, many with specialized functions requiring unique combinations of proteins and protein structures such as glycoproteins (a protein combined with a carbohydrate) to accomplish the required task or tasks a specialized cell is designed to perform. Forms of glycoproteins are known to be utilized as cell-surface receptors.

Viruses are obligate parasites. Viruses simply represent a carrier of genetic material and by themselves viruses are unable to replicate or carry on any form of biologic function outside their host cell. Viruses are generally comprised of one or more shells constructed of one or more layers of protein or lipid material, and inside the outer shell or shells, a virus carries a genetic payload that represents the instruction code necessary to replicate the virus, and protein enzymes to help facilitate the genetic payload in the function of replicating copies of the virus once the genetic payload has been delivered to a host cell. Located on the outer shell or envelope of a virus are probes. The function of a virus's probes is to locate and engage a host cell's receptors. The virus's surface probes are designed to detect, make contact with and functionally engage one or more receptors located on the exterior of a cell type that will offer the virus the proper environment in which to construct copies of itself. A host cell is a cell that provides the virus the proper biochemical machinery for the virus to successfully replicate itself.

Protected by the outer coat generally comprised of an envelope or capsid or envelope and capsid, viruses carry a genetic payload in the form of deoxyribonucleic acid (DNA) or ribonucleic acid (RNA). Once a virus's exterior probes locate and functionally engage the surface receptor or receptors on a host cell, the virus inserts its genetic payload into the interior of the host cell. In the event a virus is carrying a DNA payload, the virus's DNA travels to the host cell's nucleus and is known to become inserted into the host cell's own native DNA. In the case where a virus is carrying its genetic payload as RNA, the virus inserts the RNA payload into the host cell and may also insert one or more enzymes to facilitate the RNA being utilized properly to replicate copies of the virus. Once inside the host cell, some species of virus facilitate their RNA being converted to DNA. Once the viral RNA has been converted to DNA, the virus's DNA travels to the host cell's nucleus and is known to become inserted into the host cell's native DNA. Once a virus's genetic material has been inserted into the host cell's native DNA, the virus's genetic material takes command of certain cell functions and redirects the resources of the host cell to generate copies of the virus. Other forms of RNA viruses bypass the need to use the host cell's nuclear DNA and simply utilize portions of its innate viral genome to act as messenger RNA (mRNA). RNA viruses that bypass the host cell's DNA, cause the cell, in general, to generate copies of the necessary parts of the virus directly from the virus's RNA genome. When a virus's genome directly acts as a template, then similar to the cell's messenger RNA, the virus's RNA is read by the cell's ribosomes and proteins necessary to complete the virus's replication process are generated.

Current state of gene therapy generally refers to efforts directed toward inserting an exogenous subunit of DNA into a vehicle such as a naturally occurring virus. The vehicle is intended to insert the exogenous subunit of DNA into a target cell. The exogenous DNA subunit then migrates to the target cell's nucleus. The exogenous DNA subunit then inserts into the native DNA of the cell. This represents a permanent alteration of the cell's nuclear DNA. At some point, the nuclear transcription proteins read the exogenous DNA subunit's nucleotide coding to produce the intended cellular response. The approach described within the scope of this text involves a medically therapeutic drug as a payload versus DNA or RNA as a payload. DNA is comprised of the nucleotides adenine, thymine, guanine and cytosine. RNA is composed of the nucleotides adenine, uracil, guanine and cytosine. DNA codes acts as a template to code for the manufacture of RNA molecules. RNA acts as a template coding for the manufacture of proteins, which are composed of amino acids. A drug acts to function as a participant in a chemical reaction, as either a catalyst of the reaction or with another substance to produce one or more additional substances, these additional substances often having different properties. A safer, more effective treatment of many diseases may be approached by utilizing modified viruses as vehicles to transport a medically therapeutic drug molecules to specific cells in the body with the intent to have the drug exert an effect only on those cells to which the modified virus virions deliver the drug.

The Human Immunodeficiency Virus (HIV) is an ingeniously configured, deadly virus. Viruses, in general, have been difficult to contain and eradicate due to their being obligate parasites and the fact they tend not to carry out biologic functions outside the cell the virus has targeted as its host. An intact, individual form of a virus, as it exists outside the boundaries of a host cell, is generally referred to as a ‘virion’. The human body's immune system possesses innate mechanisms to repel viral infections once such a pathogen breaches the perimeter defenses and is recognized as an invader. If a cell is determined to be infected with a virus, neighboring cells may generate a defense response that causes neighboring cells to resist infection or if neighboring cells become infected, such cells shuts down biologic processes the virus might attempt to utilize for the purpose of replication. HIV virions possess several attributes that make them especially elusive, circumventing the immune system's routine defensive measures.

Acquired Immune Deficiency Syndrome (AIDS) occurs as a result of the number of circulating T-Helper cells declining to a point where the immune system's capacity to mount a successful response against opportunistic infectious agents is critically compromised. The number of viable T-Helper cells declines either because they become infected with the HIV virus or because they have been killed by encountering a T-Helper cell infected with HIV. When there is an insufficient population of non-HIV infected T-Helper cells to properly combat infectious agents such as Pneumocystis carinii or cytomegalovirus or other pathogens, the body becomes overwhelmed with the opportunistic infection and the patient becomes ill. In cases where the combination of the patient's compromised immune system and medical assistance in terms of synthetic antibiotics intended to combat the opportunistic pathogens, fluids, intravenous nutrition and other treatments are not sufficient to sustain life, the body succumbs to the opportunistic infection and death ensues.

The human immunodeficiency virus's outermost shell is referred to as its envelope. HIV locates its host by utilizing probes affixed to the outer surface of the envelope. The HIV virus has at least two types of glycoprotein probes attached to the outer surface of its envelope. HIV utilizes a glycoprotein probe 120 (gp 120) to locate a CD4 cell-surface receptor on a T-Helper cell. Once an HIV gp 120 probe has successfully engaged a CD4 cell surface-receptor on a T-Helper cell a conformational change occurs in the probe and a glycoprotein 41 (gp 41) probe is exposed on HIV's surface. The gp 41 probe's intent is to engage a CXCR4 or CCR5 cell-surface receptor on the same T-Helper cell. Once a gp 41 probe on the HIV virion engages a CXCR4 or CCR5 cell-surface receptor, HIV opens an access port through the T-Helper cell's outer membrane.

Once the virus procures an access port into the T-Helper cell, the HIV virion inserts into the T-Helper cell two positive strand RNA molecules approximately 9500 nucleotides in length. Inserted along with the RNA strands are the enzymes: reverse transcriptase, protease and integrase. Once the virus's genome gains access to the interior of the T-Helper cell, in the cytoplasm, the pair of RNA molecules are transformed to deoxyribonucleic acid by the reverse transcriptase enzyme. Following modification of the virus's genome to DNA, the virus's genetic information migrates to the host cell's nucleus. In the nucleus, with the assistance of the integrase protein, the virus's DNA becomes inserted into the T-Helper cell's native DNA. When the timing is appropriate, the now integrated viral DNA becomes read by a host cell's polymerase molecule and the virus's genetic information commands certain cell functions to carry out the replication process to construct copies of the human deficiency virus.

The outer layer of the HIV virion is comprised of a portion of the T-Helper cell's outer cell membrane. In the final stage of the replication process, as a copy of the HIV internal shell referred to as a capsid, which carries the HIV genome, buds through the host cell's outer membrane, the capsid acquires as its outermost shell or envelope, a wrapping of lipid bilayer, which it harvests from the host cell's outer membrane.

There is currently no vaccine or cure for AIDS. Current therapies are in the form of antiretroviral therapy. Combination therapy is often used to interfere with HIV replication inside an infected T-Helper cell. Anti-viral therapy includes nucleoside analogue reverse transcriptase inhibitors (NARTI), protease inhibitors and non-nucleoside reverse transcriptase inhibitor (NNRTI).

A means of treating AIDS could be to take the naturally occurring Human Immunodeficiency Virus (HIV) which is an RNA virus and replace HIV's innate genome with a quantity of anti-viral drug molecules. HIV already posses the means to locate and infect T-Helper cells, which act as the natural host for HIV replication. Modified HIV virions could be utilized to carry anti-viral drugs directly to infected as well as non-infected T-Helper cells. Utilizing modified HIV virions to directly insert antiviral drugs directly in a patient's T-Helper cells would prevent HIV from being able to infect a patient's T-Helper cell population and thus stop AIDS from occurring or progressing in a patient. HIV may affect other cell lines that possess CD4 cell-surface markers, so utilizing modified HIV virions that have their exterior glycoprotein probes altered to engage specific target cells in the body to directly insert antiviral drugs directly into any cell with CD4 cell-surface receptors, other than limited to just T-Helper cells, may also prevent HIV from being able to infect a patient and thus stop AIDS from occurring or progressing in a patient.

### BRIEF SUMMARY OF THE INVENTION

The medical treatment device comprised of a modified virus or virus-like structure is used as a transport medium to carry a payload consisting of a quantity of anti-viral drug molecules to T-Helper cells in the body. The modified virus or virus-like structure makes contact with a T-Helper cell by means of the virus's exterior probes or virus-like structure's exterior probes. Once the exterior probes engage the target cell's receptors, the modified virus or virus-like structure inserts into the T-Helper cell the quantity of medically therapeutic anti-viral drug molecules it is carrying.

### DETAILED DESCRIPTION

Viruses or virus-like structures can be fashioned to act as transport vehicles to carry and deliver medically therapeutic drug molecules directly to specific cells. The medically therapeutic drug carried by therapeutic modified viruses or virus-like structures would supply the cells of the body with the drug without interfering or harming other cells in the body.

Naturally occurring viruses can be altered by replacing the genetic material the virus would carry, with medically therapeutic drug molecules that would have a medically beneficial therapeutic effect on cells. The naturally occurring virus would then carry and deliver to its natural target cell the payload of medically therapeutic drug molecules.

Naturally occurring viruses can be further modified to have their naturally occurring glycoprotein surface probes replaced by glycoprotein surface probes that target specific cells in the body. Viruses modified to carry and deliver medically therapeutic drug molecules as the payload, further modified to have their glycoprotein surface probes, that cause the modified virus to engage specific cells in the body, provides a device whereby specific cells in the body can be targeted and this device embodies a means of providing to a specific type of cell in the body a drug to participate in chemical reactions with the intent to accomplish a medically therapeutic outcome.

Virus-like structures can be constructed with similar physical characteristics to naturally occurring viruses and be fashioned to carry medically therapeutic drug molecules as the payload and have located on the surface glycoprotein probes that engage specific cells in the body. Viruses-like structures carrying medically therapeutic drug molecules as the payload, constructed to have their glycoprotein surface probes engage specific cells in the body, and deliver to those specific cells the drug the virus-like structures carry provides a device whereby specific cells in the body can be targeted and this device embodies a means of providing to a specific type of cell in the body a drug to participate in chemical reactions with the intent to accomplish a medically therapeutic outcome. The advantage of a virus-like structure is that the physical dimensions of the virus-like structure can be adjusted to accommodate variations in the physical size of the payload of medically therapeutic drug molecules, yet maintain a means of engaging targeted cells in the body and delivering to those targeted cells the drug molecules required to accomplish the desired medical therapeutic outcome. A second advantage of utilizing virus-like structures is to be able to change the surface characteristics of the transport vehicle to prevent the body's immune system from reacting to the presence of the therapeutic modified virus and destroying the modified virus before it is able to deliver the payload it carries to the cells it has been designed to target. HIV utilizes an exterior envelope comprised of the surface membrane of its host, the T-Helper cell, which acts as a disguise to fool the body's immune system detection resources. Virus-like structures could be fashioned, similar to HIV, to have as an exterior envelope a surface that resembles a cell's outer membrane in order to avoid detection by the body's immune system to improve survivability and functionality of the virus-like structure.

Replicating viruses and constructing viruses to carry DNA payloads is a form of manufacturing technology that has already been well established and is in use facilitating the concept of gene therapy. Replicating viruses and designing these viruses to carry drug as the genetic payload would incorporate similar techniques as already proven useful in current DNA gene therapy technologies.

To carry out the process to manufacture a modified medically therapeutic Human Immunodeficiency Virus virions, DNA or RNA that would code for the general physical structures of the Human Immunodeficiency Virus virion would be inserted into a host. The host may include devices such as a host cell or a hybrid host cell. The host may utilize DNA or RNA or a combination of genetic instructions in order to accomplish the construction of medically therapeutic virus virions. The DNA or messenger RNA molecules to create the medically therapeutic virus virions would direct the cells to generate copies of the medically therapeutic Human Immunodeficiency Virus virions carrying a medically therapeutic drug payload. In some cases DNA or messenger RNA would be inserted into the host that would be coded to cause the production of surface probes that would be affixed to the surface of the Human Immunodeficiency Virus virions that would target the surface receptors on specific cells in the body other than the T-Helper cells the Human Immunodeficiency Virus virions naturally targets. DNA or messenger RNA would direct the host to generate copies of the medically therapeutic drug molecules that would provide a therapeutic action, or alternatively the medically therapeutic drug molecules would be artificially introduced into the host; these medically therapeutic drug molecules would take the place of the Human Immunodeficiency Virus's innate genome as its payload. The medical treatment form of the Human Immunodeficiency Virus carrying medically therapeutic drug molecules would be produced, assembled and released from a host. Virus-like structures would be generated in similar fashion using a host such as host-cells or hybrid host cells. The copies of the medically therapeutic Human Immunodeficiency Virus virions or virus-like structures, upon exiting the host, would be collected, stored and utilized as a medical treatment as necessary.

A means of treating AIDS could be to take the naturally occurring Human Immunodeficiency Virus (HIV) which is an RNA virus and replace HIV's innate genome with a quantity of anti-viral drug molecules. Anti-viral therapies include nucleoside analogue reverse transcriptase inhibitors (NARTI), protease inhibitors or non-nucleoside reverse transcriptase inhibitor (NNRTI). HIV already posses the means to locate and infect T-Helper cells, which act as the natural host for HIV replication. Modified HIV virions could be utilized to carry anti-viral drugs directly to infected as well as non-infected T-Helper cells. Utilizing modified HIV virions to directly insert antiviral drugs directly in a patient's T-Helper cells would prevent HIV from being able to infect a patient's T-Helper cell population and thus stop AIDS from occurring or progressing in a patient. HIV may affect other cell lines that possess CD4 cell-surface markers, so utilizing modified HIV virions that have their exterior glycoprotein probes altered to engage specific target cells in the body to directly insert antiviral drugs directly into any cell with CD4 cell-surface receptors, other than limited to T-Helper cells, may also prevent HIV from being able to infect a patient and thus stop AIDS from occurring or progressing in a patient.

The modified Human Immunodeficiency Virus virions and virus-like structures would be incapable of replication on their own due to the fact that the messenger RNA that would code for the replication process to produce copies of the virus or virus-like structure would not be present in the modified form of the Human Immunodeficiency Virus virions or virus-like structure.

In review, the medical device described in this text includes taking a naturally occurring Human Immunodeficiency Virus virion and altering its payload so that it transports medically therapeutic drug molecules to a T-Helper cell it is naturally designed to infect, but instead of delivering its own genetic payload, it delivers the medically therapeutic drug molecules it is carrying, and the medical device described in this text includes taking a naturally occurring Human Immunodeficiency Virus virion and altering its payload so that it carries medically therapeutic drug molecules to cells and alter the virus's glycoprotein probes so that it is capable of infecting specific targeted cells, but instead of delivering its own genetic payload, it delivers the medically therapeutic drug molecules it is carrying to a specific target cell, and the medical device described in this text includes taking a virus-like structure, which carries medically therapeutic drug molecules to cells, affixed to the surface glycoprotein probes so that it is capable of delivering medically therapeutic drug molecules it is carrying to specific target cells.

The described medical device includes a quantity of modified Human Immunodeficiency Virus virions or virus-like structures would be introduced into a patient's blood stream or tissues so that the modified virus could deliver the medially therapeutic anti-viral drug payload that it carries to T-Helper cells in the body.

The described medical device includes a quantity of modified Human Immunodeficiency Virus virions or virus-like structures introduced into a patient's blood stream or tissues so that the modified virus or virus-like structure could deliver the medially therapeutic anti-viral drug payload that it carries to the specific target cells in the body that the modified Human Immunodeficiency Virus's or virus-like structure's exterior glycoprotein probes are constructed to engage.

By utilizing the described method to provide the T-Helper cells of the body with therapeutic anti-viral drug molecules in an effort to enhancing the capacity of T-Helper cells to prevent infection by HIV and in cells already infected with HIV to combat HIV to prevent the virus from using the T-Helper cell as a host to manufacture copies of the virus, which will result the betterment of medical management for patients infected with HIV who may progress to AIDS or already have AIDS.

### DRAWING

None.

What is claimed:

1. A medical treatment device for inserting a quantity of medically therapeutic drug molecules into specific cells of the body comprising:
(a) a quantity of modified virus virions generated for the purpose of transporting a quantity of said medically therapeutic drug molecules,
(b) the quantity of said modified virus virions having a quantity of glycoprotein probes affixed to their surface, said glycoprotein probes constructed in a manner to target said specific cells in said body,
(c) said glycoprotein probes capable of engaging specific cell-surface receptors on said cells,
(d) once said glycoprotein probes have successfully engaged cell-surface receptors on said cells, the quantity of said modified virus virions deliver into said cells a quantity of said medically therapeutic drug molecules said modified virus virions are carrying,
whereby the chemical action of said medically therapeutic drug molecules will be available in said cells for the purpose of treating a medical disease,
whereby the adverse side effects of said medically therapeutic drug molecules will be minimized by the action of said drug being delivered specifically to said cells that would benefit from the presence of said drug rather than exposing the cells of the body as a whole to the effects of said medically therapeutic drug molecules,
whereby to achieve a therapeutic effect of said drug a much lower dose of said drug in comparison to conventional dosing will be required due to said drug molecules being delivered directly to said cells that would benefit from the chemical reaction said drug molecules would participate in while present in said cells resulting in a lower frequency of drug toxicity experienced by said body.

2. The medical treatment device in claim 1 wherein said modified virus virions selected from the group consisting of naturally occurring virus virions whose payload has been altered to carry a quantity of medically therapeutic drug molecules, naturally occurring virus virions whose payload has been altered to carry a quantity of medically therapeutic drug molecules said virus virions capable of delivering said quantity of medically therapeutic drug molecules which the surface glycoprotein probes have been altered in a manner the glycoprotein probes are fashioned to engage specific cells in said body, and virus-like structures constructed to resemble naturally occurring virus virions said virus-like structures capable of carrying a quantity of medically therapeutic drug molecules said virus-like structures constructed with glycoprotein probes fashioned to engage specific cells in said body said virus-like structures capable of delivering said quantity of medically therapeutic drug molecules to said specific cells in said body.

3. The medical treatment device in claim 1 wherein said specific cells selected from the group consisting of cells with CD4 cell-surface receptors, cells comprising a cancer, cells comprising a malignancy, cells comprising a tumor, cells comprising synovial tissues surrounding a joint, cells comprising synovial tissues surrounding a tendon, cells comprising the muscles, cells comprising the brain, cells comprising the heart, cells comprising the pancreas, cells comprising the endocrine glands, cells comprising the dermis, cells comprising the mucosa, cells comprising the gastroenteric tract, cells comprising the renal system, cells comprising the skeletal structures, cells comprising the pulmonary system, cells comprising the nervous system, cells comprising the immune system, cells comprising the sex organs, cells comprising the connective tissues, cells comprising the spleen, cells comprising the eyes, cells comprising the reticuloendothelial system, and cells comprising the liver.

4. The medical treatment device in claim 1 wherein said body is comprised of the physical features of the human body.

5. A medical treatment device for inserting a quantity of medically therapeutic drug molecules into cells with CD4 cell-surface receptors in the body comprising:
(a) a quantity of modified virus virions generated for the purpose of transporting a quantity of said medically therapeutic drug molecules,
(b) the quantity of said modified virus virions having a quantity of glycoprotein probes affixed to their surface, said glycoprotein probes constructed in a manner to target cells with CD4 cell-surface receptors in said body,
(c) said glycoprotein probes capable of engaging specific cell-surface receptors on said cells with CD4 cell-surface receptors,
(d) once said glycoprotein probes have successfully engaged cell-surface receptors on said cells with CD4 cell-surface receptors, the quantity of said modified virus virions deliver into said cells with CD4 cell-surface receptors a quantity of said medically therapeutic drug molecules said modified virus virions are carrying,
whereby the chemical action of said medically therapeutic drug molecules will be available in said cells with CD4 cell-surface receptors to treat a medical disease,
whereby the adverse side effects of said medically therapeutic drug molecules will be minimized by the action of said drug being delivered specifically to said cells with CD4 cell-surface receptors that would benefit from the presence of said drug rather than exposing the cells of the body as a whole to the effects of said medically therapeutic drug molecules,
whereby to achieve a therapeutic effect of said drug molecules a much lower dose of said drug will be required due to said drug molecules being delivered directly to said cells with CD4 cell-surface receptors that would benefit from the chemical reaction said drug molecules would participate in while present in said cells with CD4 cell-surface receptors resulting in a lower frequency of drug toxicity experienced by said body.

6. The medical treatment device in claim 5 wherein said medically therapeutic drug molecules is a quantity of medically therapeutic anti-viral drug molecules,
whereby said cells with CD4 cell-surface receptors are made capable of repelling a viral infection by having inserted into the cell a quantity of medically therapeutic anti-viral drug molecules.

7. The medical treatment device in claim 5 wherein said medically therapeutic drug molecules is selected from the group consisting of nucleoside analogue reverse transcriptase inhibitors (NARTI), protease inhibitors and non-nucleoside reverse transcriptase inhibitors (NNRTI),
whereby said cells with CD4 cell-surface receptors are made capable of repelling a viral infection by having inserted into said cells with CD4 cell-surface receptors a quantity of medically therapeutic anti-viral drug molecules.

8. The medical treatment device in claim 5 wherein said modified virus virions are a modified form of Human Immunodeficiency Virus virions.

9. The medical treatment device in claim 8 wherein said modified form of Human Immunodeficiency Virus virions have been altered in such a manner to replace the innate ribonucleic acid genome of the virus with a quantity of anti-viral drug molecules,
whereby said cells with CD4 cell-surface receptors are made capable of repelling a viral infection by having inserted into said cells with CD4 cell-surface receptors a quantity of medically therapeutic anti-viral drug molecules.

10. The medical treatment device in claim 8 wherein said modified form of Human Immunodeficiency Virus virions have been altered in such a manner to replace the innate replication enzymes carried in the core of the virus with a quantity of anti-viral drug molecules,
whereby said cells with CD4 cell-surface receptors are made capable of repelling a viral infection by having inserted into said cells with CD4 cell-surface receptors a quantity of medically therapeutic anti-viral drug molecules.

11. The medical treatment device in claim 5 wherein said modified virus virions selected from the group consisting of naturally occurring virus virions whose payload has been altered to carry a quantity of medically therapeutic drug molecules, naturally occurring virus virions whose payload has been altered to carry a quantity of medically therapeutic drug molecules said virus virions capable of delivering said quantity of medically therapeutic drug which the surface glycoprotein probes have been altered in a manner the glycoprotein probes are fashioned to engage cells with CD4 cell-surface receptors in said body, and virus-like structures constructed to resemble naturally occurring virus virions said virus-like structures capable of carrying a quantity of medically therapeutic drug molecules said virus-like structures constructed with glycoprotein probes fashioned to engage cells with CD4 cell-surface receptors in said body said virus-like structures capable of delivering said quantity of medically therapeutic drug molecules to said cells with CD4 cell-surface receptors in said body.

12. The medical treatment device in claim 5 wherein said body is comprised of the physical features of the human body.

13. A medical treatment device for inserting a quantity of medically therapeutic drug molecules into T-Helper cells of the body comprising:
(a) a quantity of modified virus virions generated for the purpose of transporting a quantity of said medically therapeutic drug molecules,
(b) the quantity of said modified virus virions having a quantity of glycoprotein probes affixed to their surface, said glycoprotein probes constructed in a manner to target said T-Helper cells in said body,
(c) said glycoprotein probes capable of engaging specific cell-surface receptors on said T-Helper cells,
(d) once said glycoprotein probes have successfully engaged cell-surface receptors on said T-Helper cells, the quantity of modified virus virions deliver into said T-Helper cells a quantity of said medically therapeutic drug molecules said modified virus virions are carrying,
whereby the chemical action of said medically therapeutic drug molecules will be available in said T-Helper cells for the purposes of treating a medical disease,
whereby the adverse side effects of said medically therapeutic drug molecules will be minimized by the action of said drug being delivered specifically to said T-Helper cells that would benefit from the presence of said drug rather than exposing the cells of the body as a whole to the effects of said medically therapeutic drug molecules,
whereby to achieve a therapeutic effect of said drug a much lower dose of said drug in comparison to conventional dosing will be required due to said drug molecules being delivered directly to said T-Helper cells that would benefit from the chemical reaction said drug would participate in while present in said T-Helper cells resulting in a lower frequency of drug toxicity experienced by said body.

14. The medical treatment device in claim 13 wherein said medically therapeutic drug molecules is a quantity of medically therapeutic anti-viral drug molecules, whereby said T-Helper cells are made capable of repelling a viral infection by having inserted into said T-Helper cells a quantity of medically therapeutic anti-viral drug molecules.

15. The medical treatment device in claim 13 wherein said medically therapeutic drug molecules is selected from the group consisting of nucleoside analogue reverse transcriptase inhibitors (NARTI), protease inhibitors and non-nucleoside reverse transcriptase inhibitors (NNRTI),
whereby said T-Helper cells are made capable of repelling a viral infection by having inserted into said T-Helper cells a quantity of medically therapeutic anti-viral drug molecules.

16. The medical treatment device in claim 13 wherein said modified virus virions is a modified form of Human Immunodeficiency Virus virions.

17. The medical treatment device in claim 16 wherein said modified form of Human Immunodeficiency Virus virions have been altered in such a manner to replace the innate ribonucleic acid genome carried in the core of the virus with a quantity of anti-viral drug molecules,
whereby said T-Helper cells are made capable of repelling a viral infection by having inserted into said T-Helper cells a quantity of medically therapeutic anti-viral drug molecules.

18. The medical treatment device in claim 16 wherein said modified form of Human Immunodeficiency Virus virions have been altered in such a manner to replace the innate replication enzymes carried in the core of the virus with a quantity of anti-viral drug molecules,
whereby said T-Helper cells are made capable of repelling a viral infection by having inserted into said T-Helper cells a quantity of medically therapeutic anti-viral drug molecules.

19. The medical treatment device in claim 13 wherein said modified virus virions selected from the group consisting of naturally occurring virus virions whose payload has been altered to carry a quantity of medically therapeutic drug molecules, naturally occurring virus virions whose payload has been altered to carry a quantity of medically therapeutic drug molecules said virus virions capable of delivering said quantity of medically therapeutic drug molecules which the surface glycoprotein probes have been altered in a manner the glycoprotein probes are fashioned to engage T-Helper cells in said body, and virus-like structures constructed to resemble naturally occurring virus virions said virus-like structures capable of carrying a quantity of medically therapeutic drug molecules said virus-like structures constructed with glycoprotein probes fashioned to engage T-Helper cells in said body said virus-like structures capable of delivering said quantity of medically therapeutic drug molecules to said T-Helper cells in said body.

20. The medical treatment device in claim 13 wherein said body is comprised of the physical features of the human body.
